(19) United States
(12) Patent Application Publication
(10) Pub. No.: US 2026/0232454 A1
(43) Pub. Date: Aug. 13, 2026

(54) LINER LOCKING SYSTEMS AND ASSEMBLY METHODS FOR ARTHROPLASTY IMPLANT SYSTEMS

(71) Applicant: ARTHREX, INC., Naples, FL (US)

(72) Inventors: Michael Alan Knight, Naples, FL (US); Rudraksh Khosla, Naples, FL (US); Dean Acker, Naples, FL (US); Tyler Clevett, Bonita Springs, FL (US); Gregory Guederian, Naples, FL (US); Jason Schultz, Naples, FL (US); Mark Wise, Warsaw, IN (US)

(21) Appl. No.: 19/534,885

(22) Filed: Feb. 10, 2026

Related U.S. Application Data
(60) Provisional application No. 63/757,044, filed on Feb. 11, 2025.

(51) Int. Cl. A61F 2/40; A61F 2/30

(52) U.S. Cl. CPC A61F 2/4014; A61F 2002/30507

(57) ABSTRACT

Arthroplasty implant systems and methods are provided for restoring the functionality of a joint. The arthroplasty implant systems may include a liner locking system for assembling a liner to a joint adapter. The liner locking system may include a twist on design or a collet design.

### CROSS-REFERENCE TO RELATED APPLICATIONS

This disclosure claims the benefit of U.S. Provisional Application No. 63/757,044, filed Feb. 11, 2025, which is incorporated herein by reference in its entirety.

### BACKGROUND

This disclosure relates to the field of arthroplasty, and more particularly to arthroplasty implant systems that include liner locking systems for establishing mating connections between arthroplasty implant components.

Many bones of the human musculoskeletal system include articular surfaces. The articular surfaces cooperate to facilitate different types and degrees of joint movement. The articular surfaces can erode or experience bone loss over time due to repeated use or wear, thereby causing joint instability and pain.

Arthroplasty is an orthopedic surgical procedure performed to repair or replace joints that exhibit degenerative bone deficiencies. Bone deficiencies may occur along the articular surfaces of bone. Some arthroplasty procedures utilize one or more implants to repair the articular surfaces.

### SUMMARY

This disclosure relates to arthroplasty implant systems and methods designed for restoring functionality to a joint. The arthroplasty implant systems may include an implant assembly that includes a joint adapter and a liner.

An exemplary implant assembly for an arthroplasty implant system may include, inter alia, a joint adapter including a boss, and a liner including a locking tab that is configured to flex between a first position and a second position relative to the boss to lock the liner to the joint adapter.

An exemplary implant assembly for an arthroplasty implant system may include, inter alia, a joint adapter including a collet, a liner including a groove configured to receive the collet, and a locking screw insertable into the collet for locking the liner to the joint adapter.

The embodiments, examples, and alternatives of the preceding paragraphs, the claims, or the following description and drawings, including any of their various aspects or respective individual features, may be taken independently or in any combination. Features described in connection with one embodiment are applicable to all embodiments unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates an exemplary arthroplasty implant system.

FIG. 2 is an exploded view of an exemplary humeral implant assembly of an arthroplasty implant system.

FIG. 3 is another exploded view of the humeral implant assembly of FIG. 2.

FIG. 4 illustrates an unlocked position of a liner relative to a joint adapter of the humeral implant assembly illustrated in FIGS. 2-3.

FIG. 5 illustrates a locked position of the liner relative to the joint adapter of the humeral implant assembly illustrated in FIG. 4.

FIG. 6 illustrates an exemplary liner of a humeral implant assembly.

FIG. 7 illustrates another exemplary humeral implant assembly.

FIG. 8 is an exploded view of the humeral implant assembly of FIG. 7.

FIG. 9 is a cross-sectional view of the humeral implant assembly of FIG. 7.

FIG. 10 is a partial exploded view of the humeral implant assembly of FIG. 7.

FIG. 11 illustrates yet another exemplary humeral implant assembly.

FIG. 12 illustrates an exemplary liner of the humeral implant assembly of FIG. 11.

### DETAILED DESCRIPTION

This disclosure describes arthroplasty implant systems and methods for restoring the functionality of a joint. The arthroplasty implant systems may include features for establishing a mating connection between a liner and a joint adapter of an implant assembly of the system.

An exemplary implant assembly for an arthroplasty implant system may include a joint adapter including a boss, and a liner including a locking tab that is configured to flex between a first position and a second position relative to the boss to lock the liner to the joint adapter.

In any further embodiment, the implant assembly is a humeral implant assembly.

In any further embodiment, the joint adapter includes a flange portion and a cylindrical base portion, and the boss protrudes inwardly from an inner diameter wall of the cylindrical base portion.

In any further embodiment, a clearance space extends between the boss and a floor of the cylindrical base portion.

In any further embodiment, a retention ledge of the liner is accommodated within the clearance space when the liner is locked to the joint adapter.

In any further embodiment, the locking tab is positioned adjacent to a leading edge of the retention ledge.

In any further embodiment, the liner includes an articular portion and an engagement portion, and the locking tab protrudes outwardly from an outer diameter wall of the engagement portion.

In any further embodiment, a retention ledge protrudes outwardly from the outer diameter wall at a location that is distal to the locking tab.

In any further embodiment, an exposed section of the outer diameter wall extends between the retention ledge and a second retention ledge of the engagement portion.

In any further embodiment, the locking tab is configured to flex between the first position and the second position in response to rotating the liner relative to the joint adapter.

An exemplary implant assembly for an arthroplasty implant system may include a joint adapter including a collet, a liner including a groove configured to receive the collet, and a locking screw insertable into the collet for locking the liner to the joint adapter.

In any further embodiment, the collet includes a plurality of flexible arms.

In any further embodiment, the plurality of flexible arms flex radially outwardly as the locking screw is inserted into the collet.

In any further embodiment, a transverse tab portion of each of the plurality of flexible arms is movable further into a proximal groove section of the groove as the locking screw is inserted into the collet.

In any further embodiment, the transverse tab portion extends over a circumferential ledge of the proximal groove section.

In any further embodiment, the groove is a stepped groove.

In any further embodiment, the stepped groove includes a proximal groove section having a first diameter and a distal groove portion having a second diameter that is smaller than the first diameter.

In any further embodiment, the locking screw is insertable into an opening formed in a screw housing of the joint adapter.

In any further embodiment, the opening is threaded for engaging a threaded portion of the locking screw.

In any further embodiment, the implant assembly is a humeral implant assembly.

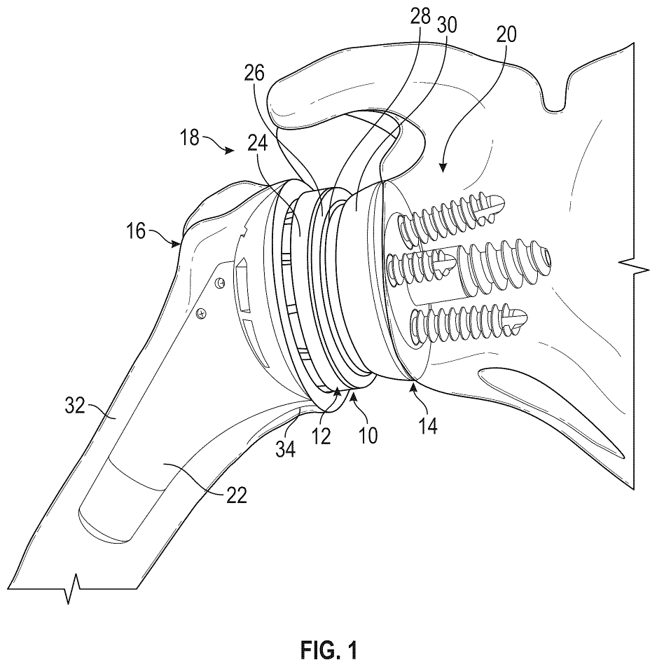

FIG. 1 illustrates an arthroplasty implant system 10 that includes a humeral implant assembly 12 and a glenoid implant assembly 14. The humeral implant assembly 12 may be implanted within a humerus 16 of a shoulder joint 18 to aid in reconstructing the native articular surface of the humerus 16 and/or restoring the functionality (e.g., range of motion, stability, etc.) of the shoulder joint 18. The glenoid implant assembly 14 may be implanted within a glenoid 20 of the shoulder joint 18 and may be configured to interface with the humeral implant assembly 12 for restoring functionality to the shoulder joint 18.

Although the teachings of this disclosure are described with specific reference to the shoulder joint 18, the teachings of this disclosure are not intended to be limited to any particular joint or bone of the human musculoskeletal system. The teachings of this disclosure could be applicable to any other joint, such as the hip joint, for example.

The humeral implant assembly 12 may include a stem portion 22, a joint adapter 24 that is connectable to the stem portion 22, and a liner 26 that is connectible to the joint adapter 24. The liner 26 may include an articular surface 28 that is configured to interface with an articular implant 30 of the glenoid implant assembly 14. For reverse shoulder arthroplasty procedures, the articular surface 28 may be a concave articular surface for providing a socket portion of the shoulder joint 18, which is the opposite of the native anatomy, and for anatomic shoulder arthroplasty procedures, the articular surface 28 may be a convex articular surface (not shown) for providing a ball portion of the shoulder joint 18.

In the illustrated embodiment, a humeral head of the humerus 16 has been resected, and thus the native articular component of the humerus 16 is removed in order to prepare the humerus 16 for receiving the humeral implant assembly 12. After the humerus 16 has been appropriately prepared, the stem portion 22 may be inserted into a diaphysis 32 of the humerus 16, and the joint adapter 24 may be positioned within a metaphysis 34 of the humerus 16. The joint adapter 24, which can alternatively be referred to as a cup or a spacer of the humeral implant assembly 12, may be configured to establish a convertible platform for receiving the liner 26.

In an embodiment, the joint adapter 24 is constructed of a metallic material (e.g., a titanium alloy), and the liner 26 is constructed of a plastic material (e.g., polyethylene). However, other materials or combinations of materials could alternatively be utilized within the scope of this disclosure.

In order to achieve a desired level of joint stability for a particular patient, a surgeon must often optimize the respective sizes of the liner 26 and the articular implant 30. This optimization can sometimes require increasing the size of articular implant 30 relative to the liner 26 by one size (referred to as a single jump) or even two sizes (referred to as a double jump). This oversizing typically moves the articular surface 28 of the liner 26 further medially, thereby leading to increased loads at the mating interface between the liner 26 and the joint adapter 24 that result from lengthening the lever arm established by the arthroplasty implant system 10. This disclosure is therefore directed to liner locking systems that provide for the ability to withstand increased loads, such as when utilizing a double jump sizing, for example.

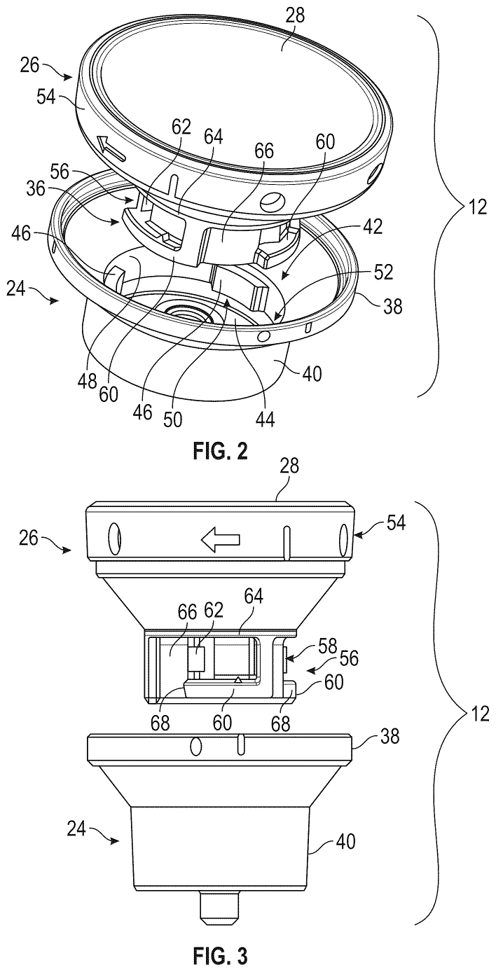

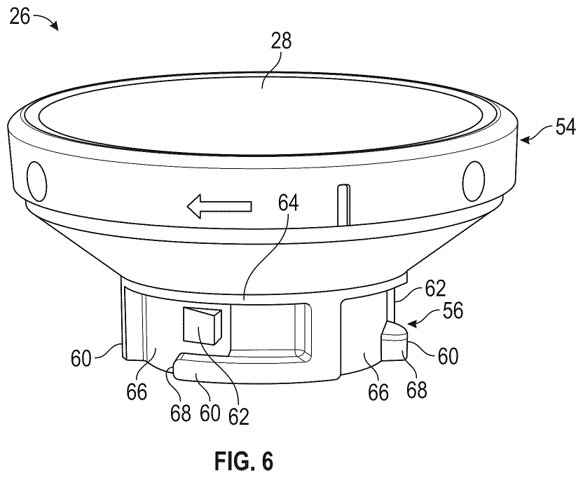

FIGS. 2-6 illustrate additional features associated with the humeral implant assembly 12 of the arthroplasty implant system 10 of FIG. 1. The humeral implant assembly 12 may include a liner locking system 36 for removably connecting and locking the liner 26 to the joint adapter 24. As further discussed below, the liner locking system 36 may be established by a combination of features provided by both the joint adapter 24 and the liner 26.

The joint adapter 24 of the humeral implant assembly 12 may include a flange portion 38 located at a top or proximal side of the joint adapter 24, and a cylindrical base portion 40 located at a bottom or distal side of the joint adapter 24. An outer diameter of the flange portion 38 may be greater than an outer diameter of the cylindrical base portion 40.

A receiving cavity 42 (best shown in FIG. 2) of the joint adapter 24 may be configured to receive the liner 26 for securing the liner 26 to the joint adapter 24. The receiving cavity 42 may be circumscribed by the flange portion 38 and the cylindrical base portion 40. The receiving cavity 42 may extend inwardly from the flange portion 38 to a floor 44 of the cylindrical base portion 40. The floor 44 may establish an inner surface of the cylindrical base portion 40.

The receiving cavity 42 may provide an inlay design in which a majority of the joint adapter 24 is disposed inside the humerus 16 when implanted. In this way, the mating connection between the liner 26 and the joint adapter 24 is also inlaid rather than exhibiting an onlay design.

One or more bosses 46 may protrude inwardly from an inner diameter wall 48 of the cylindrical base portion 40. The inner diameter wall 48 may circumscribe the portion of the receiving cavity 42 that extends within the cylindrical base portion 40. Each boss 46 may be spaced apart from the floor 44 such that a clearance space 50 extends between the boss 46 and the floor 44.

In an embodiment, a total of three bosses 46 protrude inwardly from the inner diameter wall 48, and each boss 46 may be circumferentially spaced apart from the other bosses 46 along the inner diameter wall 48. However, other configurations are contemplated within the scope of this disclosure. An additional clearance space 52 may extend circumferentially between adjacent bosses 46 of the cylindrical base portion 40 of the joint adapter 24.

In an embodiment, each boss 46 is rectangular shaped. However, other configurations are contemplated within the scope of this disclosure.

The liner 26 of the humeral implant assembly 12 may include an articular portion 54 located at a top or proximal side of the liner 26 and an engagement portion 56 located at a bottom or distal side of the liner 26. An outer diameter of the articular portion 54 may be larger than an outer diameter of the engagement portion 56.

The articular portion 54 of the liner 26 may include the articular surface 28. In the illustrated embodiment, the articular surface 28 is a concave articular surface for interfacing with a glenosphere as part of a reverse shoulder arthroplasty system. However, the articular surface 28 could alternatively be a convex articular surface for performing anatomic shoulder arthroplasty procedures within the scope of this disclosure.

The engagement portion 56 of the liner 26 may include an outer diameter wall 58 and one or more retention ledges 60 that protrude outwardly from the outer diameter wall 58. In an embodiment, a total of three retention ledges 60 protrude outwardly from the outer diameter wall 58, and each retention ledge 60 may be circumferentially spaced apart from the other retention ledges 60 along the outer diameter wall 58. However, other configurations are contemplated within the scope of this disclosure.

An exposed section 66 of the outer diameter wall 58 may extend between adjacently positioned retention ledges 60. Each exposed section 66 may be configured to interface with one of the bosses 46 of the joint adapter 24 when the engagement portion 56 of the liner 26 is positioned within the receiving cavity 42.

The engagement portion 56 of the liner 26 may additionally include one or more locking tabs 62. One locking tab 62 may be provided adjacent to each retention ledge 60 of the engagement portion 56. In an embodiment, the locking tab 62 is positioned near a leading edge 68 of the retention ledge 60. Each locking tab 62 may protrude outwardly from the outer diameter wall 58 at a location that is vertically between the retention ledge 60 and a proximal end portion 64 of the engagement portion 56. The proximal end portion 64 may be located at the interface between the articular portion 54 and the engagement portion 56.

Each locking tab 62 may be configured to flex relative to the outer diameter wall 58. For example, each locking tab 62 may flex toward the outer diameter wall 58 when it moves into contact with another surface, such as one of the bosses 46 of the joint adapter 24, for example, when assembling the liner 26 to the joint adapter 24.

The following method may be utilized to secure and lock the liner 26 to the joint adapter 24. First, the liner 26 may be positioned above the joint adapter 24 such that the retention ledges 60 are aligned with the clearance spaces 52 and the exposed sections 66 of the outer diameter wall 58 are aligned with the bosses 46. The liner 26 may then be moved distally into the receiving cavity 42 until the engagement portion 56 contacts the floor 44 of the cylindrical base portion 40.

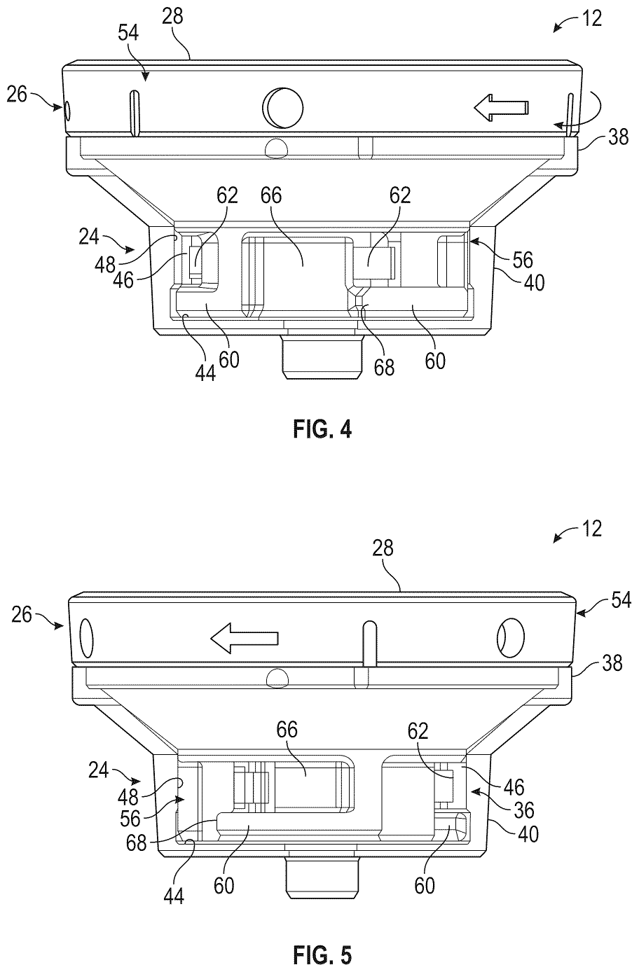

Next, the liner 26 may be rotated relative to the joint adapter 24. For example, the liner 26 may be rotated in a clockwise direction between a first position (see FIG. 4) and a second position (see FIG. 5) relative to the joint adapter 24. Rotation of the liner 26 between the first and second positions causes the retention ledges 60 to move into the clearance spaces 50 that extend between the floor 44 and the bosses 46. Meanwhile, the locking tabs 62 may flex inwardly toward the outer diameter wall 58 as they are moved into engagement with the bosses 46 of the joint adapter 24 during the rotation. Once the locking tabs 62 clear the opposite side of the bosses 46 during the rotation (best shown in FIG. 5), the locking tabs 62 may disengage from the bosses 46 and thereby flex or snap outwardly back to their original positions. At this stage, the liner 26 is locked to the joint adapter 24.

This embodiment of the liner locking system 36 provides a “twist on design” for locking the liner 26 relative to the joint adapter 24. Unlike traditional liner designs, the liner 26 can be assembled to the joint adapter 24 without requiring any impaction forces.

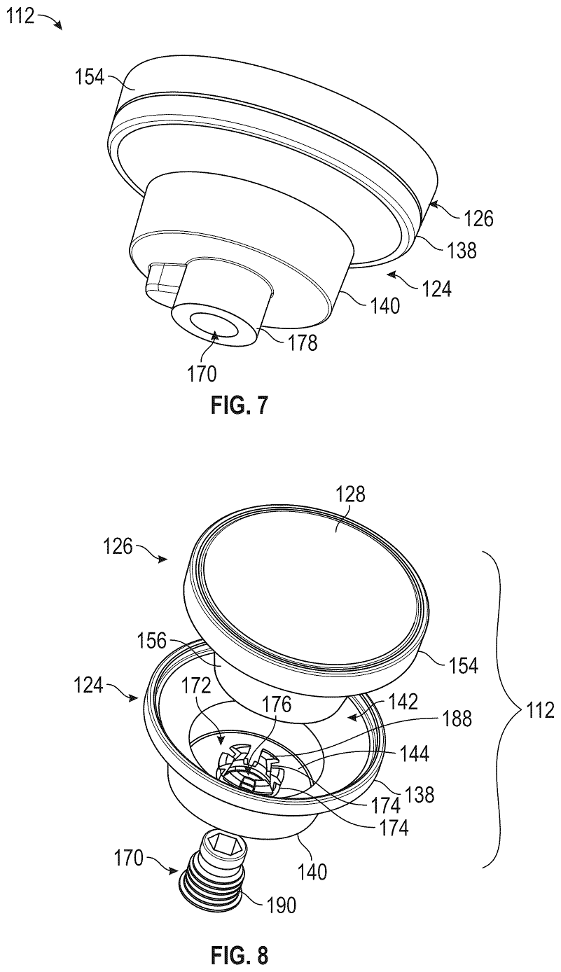

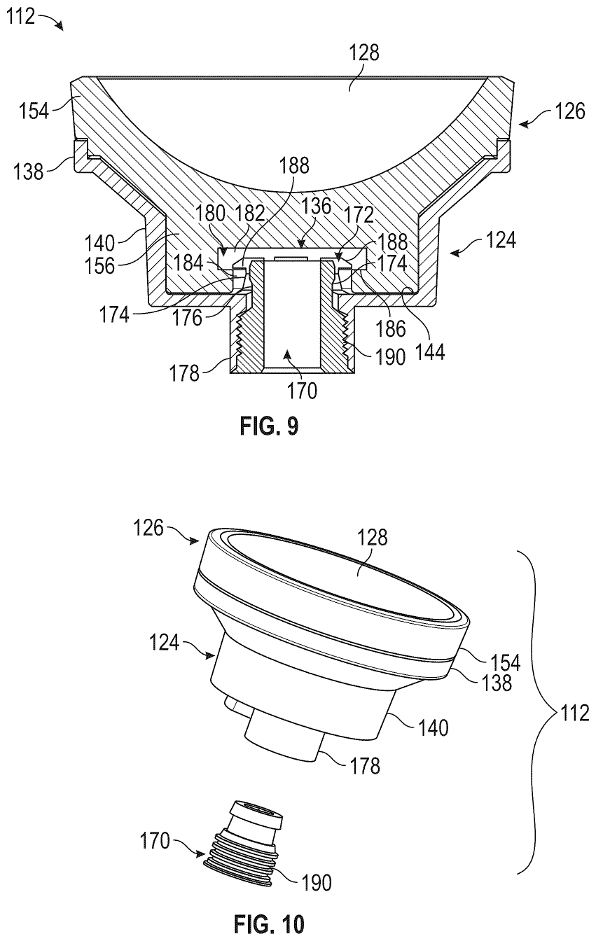

FIGS. 7-10 illustrate another exemplary humeral implant assembly 112 that can be utilized as part of an arthroplasty implant system, such as the arthroplasty implant system 10 of FIG. 1, for example. The humeral implant assembly 112 may embody a three-piece design that includes a joint adapter 124, a liner 126, and a locking screw 170. The humeral implant assembly 112 may further include a liner locking system 136 (best seen in FIG. 9) for removably connecting and locking the liner 126 to the joint adapter 124. As further discussed below, the liner locking system 136 may be established by a combination of features provided by both the joint adapter 124, the liner 126, and the locking screw 170.

The joint adapter 124 may include a flange portion 138 located at a top or proximal side of the joint adapter 124, a screw housing 178 located at a bottom or distal side of the joint adapter 124, and a cylindrical base portion 140 located between the flange portion 138 and the screw housing 178. An outer diameter of the flange portion 138 may be larger than an outer diameter of each of the cylindrical base portion 140 and the screw housing 178. The outer diameter of the cylindrical base portion 140 may be larger than the outer diameter of the screw housing 178.

A receiving cavity 142 (see FIG. 8) of the joint adapter 124 may be configured to receive the liner 126 for securing the liner 126 to the joint adapter 124. The receiving cavity 142 may be circumscribed by the flange portion 138 and the cylindrical base portion 140. The receiving cavity 142 may extend inwardly from the flange portion 138 to a floor 144 of the cylindrical base portion 140. The floor 144 may establish an inner surface of the cylindrical base portion 140.

The receiving cavity 142 may provide an inlay design in which a majority of the joint adapter 124 is disposed inside the humerus 16 when implanted. In this way, the mating connection between the liner 126 and the joint adapter 124 is also inlaid rather than exhibiting an onlay design.

A collet 172 may protrude upwardly from the floor 144 of the cylindrical base portion 140. The collet 172 may include a plurality of flexible arms 174. The flexible arms 174 may be circumferentially spaced from one another and arranged about an opening 176 of the floor 144.

The opening 176 may extend through the floor 144 and through the screw housing 178 of the cylindrical base portion 140. The opening 176 may be sized to receive the locking screw 170. The portion of the opening 176 extending through the screw housing 178 may be threaded for receiving a threaded portion 190 of the locking screw 170.

The liner 126 may include an articular portion 154 located at a top or proximal side of the liner 126 and an engagement portion 156 located at a bottom or distal side of the liner 126. An outer diameter of the articular portion 154 may be larger than an outer diameter of the engagement portion 156.

The articular portion 154 of the liner 126 may include the articular surface 128. In the illustrated embodiment, the articular surface 128 is a concave articular surface for interfacing with a glenosphere as part of a reverse shoulder arthroplasty system. However, the articular surface 128 could alternatively be a convex articular surface for performing anatomic shoulder arthroplasty procedures within the scope of this disclosure.

As best shown in FIG. 9, the engagement portion 156 of the liner 126 may include a stepped groove 180 for accommodating the collet 172 of the joint adapter 124. The stepped groove 180 may include a proximal groove section 182 and a distal groove section 184 that is connected to the proximal groove section 182. The proximal groove section 182 may include a larger diameter than that of the distal groove section 184. A circumferential ledge 186 may be formed at an interface between the proximal groove section 182 and the distal groove section 184.

The following method may be utilized to secure and lock the liner 126 to the joint adapter 124. First, the liner 126 may be positioned above the joint adapter 124. The liner 126 may then be moved distally into the receiving cavity 142 until the collet 172 of the joint adapter 124 engages the stepped groove 180 of the liner 126.

Next, the locking screw 170 may be inserted through the opening 176 and into the collet 172 of the joint adapter 124. Advancement of the locking screw 170 into the collet 172 causes the flexible arms 174 of the collet 172 to flex/expand radially outwardly. Transverse tab portions 188 of the flexible arms 174 may therefore be moved further outwardly within the distal groove section 184 to a position that extends over the circumferential ledge 186 to secure the joint adapter 124 and the liner 126 together (see, e.g., FIG. 9). Once the locking screw 170 is fully inserted, the liner 126 is locked to the joint adapter 124.

This embodiment of the liner locking system 136 provides a “collet design” for locking the liner 126 relative to the joint adapter 124. Unlike traditional liner designs, the liner 126 can be assembled to the joint adapter 124 without requiring any impaction forces.

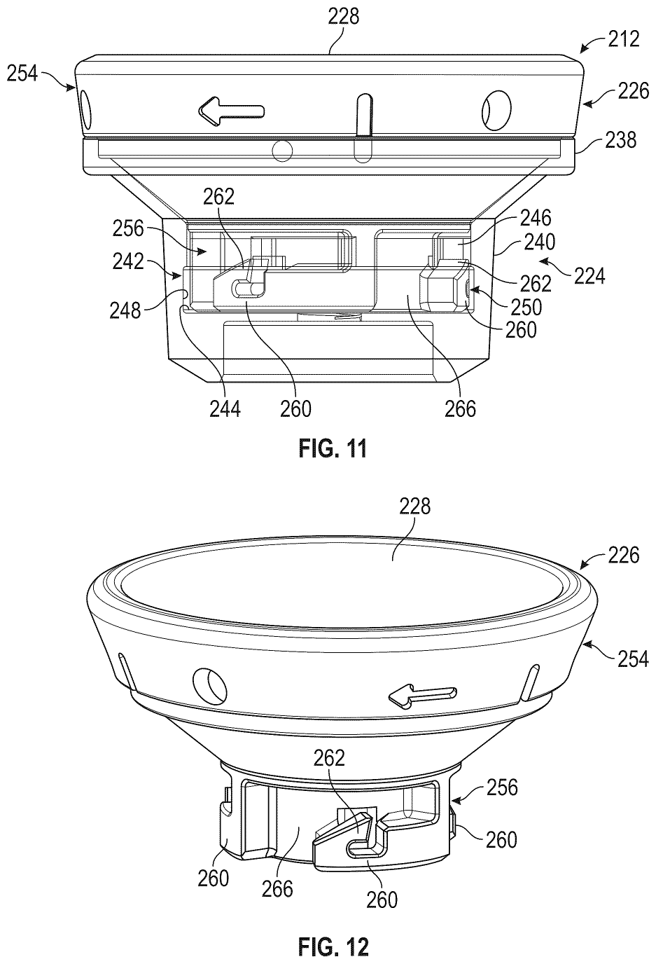

FIGS. 11-12 illustrate another exemplary humeral implant assembly 212 that can be utilized as part of an arthroplasty implant system, such as the arthroplasty implant system 10 of FIG. 1, for example. The humeral implant assembly 212 is similar to the humeral implant assembly 12 of FIGS. 2-6 and thus provides another “twist on design” for locking a liner 226 relative to a joint adapter 224. However, in this embodiment and as further described below, locking tabs 262 of the liner 226 are configured to flex axially instead of radially when assembling the liner 226 to the joint adapter 224.

The joint adapter 224 of the humeral implant assembly 212 may include a flange portion 238 located at a top or proximal side of the joint adapter 224, and a cylindrical base portion 240 located at a bottom or distal side of the joint adapter 224. An outer diameter of the flange portion 238 may be greater than an outer diameter of the cylindrical base portion 240.

A receiving cavity 242 of the joint adapter 224 may be configured to receive the liner 226 for securing the liner 226 to the joint adapter 224. The receiving cavity 242 may be circumscribed by the flange portion 238 and the cylindrical base portion 240 and may extend inwardly from the flange portion 238 to a floor 244 of the cylindrical base portion 240.

One or more bosses 246 may protrude inwardly from an inner diameter wall 248 of the cylindrical base portion 240. Each boss 246 may be spaced apart from the floor 244 to establish a clearance space 250 between the boss 246 and the floor 244.

The liner 226 of the humeral implant assembly 212 may include an articular portion 254 located at a top or proximal side of the liner 226 and an engagement portion 256 located at a bottom or distal side of the liner 226. An outer diameter of the articular portion 254 may be larger than an outer diameter of the engagement portion 256.

The articular portion 254 of the liner 226 may include the articular surface 228. The articular surface 228 may exhibit either a concave surface or a convex surface depending on the type of arthroplasty procedure being performed.

The engagement portion 256 of the liner 226 may include one or more retention ledges 260 that protrude outwardly from an outer diameter wall. An exposed section 266 of the engagement portion 256 may extend between adjacently positioned retention ledges 260. Each exposed section 266 may be configured to interface with one of the bosses 246 of the joint adapter 224 when the engagement portion 256 of the liner 226 is positioned within the receiving cavity 242.

The engagement portion 256 of the liner 226 may additionally include one or more locking tabs 262, which may be integrally formed with the retention ledges 260. Each locking tab 262 may be configured to flex relative to the respective retention ledge 260. For example, each locking tab 262 may flex axially (e.g., in the proximal toward the distal direction) when it moves into contact with another surface, such as one of the bosses 246 of the joint adapter 224, for example, when assembling the liner 226 to the joint adapter 224. Once the locking tabs 262 clear the opposite side of the bosses 246 during the rotation, the locking tabs 262 may disengage from the bosses 246 and thereby flex or snap (e.g., in the distal toward proximal direction) back to their original positions. The locking tabs 262 may therefore flex axially rather than radially in this implementation for connecting the liner 226 to the joint adapter 224.

The exemplary arthroplasty implant systems of this disclosure include implant assemblies that utilize liner locking mechanisms for establishing mating connections between the liner and adjacent structures of the assembly. The liner locking mechanisms are capable of establishing mating connections without the need for exerting any impaction forces. The liner locking mechanisms are further capable of withstanding loads seen while utilizing a double jump oversizing between the liner and an articular implant of the assembly.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. An implant assembly for an arthroplasty implant system, comprising:
a joint adapter including a boss; and
a liner including a locking tab that is configured to flex between a first position and a second position relative to the boss to lock the liner to the joint adapter.

2. The implant assembly as recited in claim 1, wherein the implant assembly is a humeral implant assembly.

3. The implant assembly as recited in claim 1, wherein the joint adapter includes a flange portion and a cylindrical base portion, and further wherein the boss protrudes inwardly from an inner diameter wall of the cylindrical base portion.

4. The implant assembly as recited in claim 3, wherein a clearance space extends between the boss and a floor of the cylindrical base portion.

5. The implant assembly as recited in claim 4, wherein a retention ledge of the liner is accommodated within the clearance space when the liner is locked to the joint adapter.

6. The implant assembly as recited in claim 5, wherein the locking tab is positioned adjacent to a leading edge of the retention ledge.

7. The implant assembly as recited in claim 1, wherein the liner includes an articular portion and an engagement portion, and further wherein the locking tab protrudes outwardly from an outer diameter wall of the engagement portion.

8. The implant assembly as recited in claim 7, wherein a retention ledge protrudes outwardly from the outer diameter wall at a location that is distal to the locking tab.

9. The implant assembly as recited in claim 8, wherein an exposed section of the outer diameter wall extends between the retention ledge and a second retention ledge of the engagement portion.

10. The implant assembly as recited in claim 1, wherein the locking tab is configured to flex between the first position and the second position in response to rotating the liner relative to the joint adapter.

11. An implant assembly for an arthroplasty implant system, comprising:
a joint adapter including a collet;
a liner including a groove configured to receive the collet; and
a locking screw insertable into the collet for locking the liner to the joint adapter.

12. The implant assembly as recited in claim 11, wherein the collet includes a plurality of flexible arms.

13. The implant assembly as recited in claim 12, wherein the plurality of flexible arms flex radially outwardly as the locking screw is inserted into the collet.

14. The implant assembly as recited in claim 13, wherein a transverse tab portion of each of the plurality of flexible arms is movable further into a proximal groove section of the groove as the locking screw is inserted into the collet.

15. The implant assembly as recited in claim 14, wherein the transverse tab portion extends over a circumferential ledge of the proximal groove section.

16. The implant assembly as recited in claim 11, wherein the groove is a stepped groove.

17. The implant assembly as recited in claim 16, wherein the stepped groove includes a proximal groove section having a first diameter and a distal groove portion having a second diameter that is smaller than the first diameter.

18. The implant assembly as recited in claim 11, wherein the locking screw is insertable into an opening formed in a screw housing of the joint adapter.

19. The implant assembly as recited in claim 18, wherein the opening is threaded for engaging a threaded portion of the locking screw.

20. The implant assembly as recited in claim 11, wherein the implant assembly is a humeral implant assembly.
